(19) United States
(12) Patent Application Publication
(10) Pub. No.: US 2024/0081777 A1
(43) Pub. Date: Mar. 14, 2024

(54) ERGONOMIC ATTACHMENTS FOR A HANDHELD ULTRASOUND PROBE

(71) Applicant: BFLY OPERATIONS, INC., Burlington, MA (US)

(72) Inventors: John Caswell, Nashua, NH (US); Timothy Hyde, Columbia, SC (US); Jason Fischman, Stamford, CT (US); Scott Rumschlag, Fenton, MI (US); Robert Schneider, Killingworth, CT (US)

(73) Assignee: BFLY OPERATIONS, INC., Burlington, MA (US)

(21) Appl. No.: 18/367,733

(22) Filed: Sep. 13, 2023

Related U.S. Application Data
(60) Provisional application No. 63/406,121, filed on Sep. 13, 2022.

(51) Int. Cl. A61B 8/00

(52) U.S. Cl. CPC A61B 8/4455

(57) ABSTRACT

Ergonomic attachments for a handheld ultrasound probe are described herein. An attachment assembly includes a plate-shaped member that includes a passage configured to accommodate the handheld ultrasound probe such that when the plate-shaped member is inserted in the attachment assembly, a portion of a transducer head of the handheld ultrasound probe protrudes from the plate-shaped member. Another attachment assembly includes a shell that attaches to the handheld ultrasound probe and partially covers the handheld ultrasound probe, and strap anchors that are each connected to the shell and are disposed at opposite ends of the shell. Another attachment includes a curved bar that is continuous between a proximal attachment flange, of the curved bar, that attaches to a first location of the handheld ultrasound probe and a distal attachment flange, of the curved bar, that attaches to a second location of the handheld ultrasound probe.

### CROSS-REFERENCE TO RELATED APPLICATIONS

This application claims the benefit of priority under 35 U S.C. § 119(e) to U.S. Provisional Patent Application Ser. No. 63/406,121, filed on Sep. 13, 2022, which is hereby incorporated by reference herein in its entirety.

### BACKGROUND

Ultrasound imaging has a wide range of applications in the medical and scientific fields for diagnosis, treatment, and studying of internal objects, within a body, such as internal organs or developing fetuses. Handheld ultrasound probes are used for ultrasound imaging in many medical settings. Typically, a handheld ultrasound probe is operated by an operator (e.g., a physician, nurse, or technician) who holds the probe in contact with a patient's body in order to acquire the desired ultrasound data and images. To achieve this, operators may need to precisely position and manipulate the probe with their hands for multiple hours during a typical workday, which may result in fatigue and potentially even stress- or strain-related injuries. Additionally, for some applications and medical situations, it may be desirable for patients to perform ultrasound imaging on themselves. However, depending on the shape of the handheld ultrasound probe, the region of the body to be imaged, and/or the health and capabilities of the patient, it may be difficult or awkward for a patient to properly achieve the desired ultrasound images via self-scanning. For example, in the case of diagnosing and monitoring congestive heart failure, there may be several scan locations and/or angles (e.g., in the axillary region), that may be difficult to self-scan for patients who are elderly, arthritic, obese, disabled, or otherwise having impaired reach or dexterity.

### SUMMARY

In general, one or more embodiments of the disclosure relate to an attachment assembly, for ergonomic handling of a handheld ultrasound probe, the attachment assembly comprising: a plate-shaped member with a proximal surface and a distal surface, the plate-shaped member comprising a passage between the proximal surface and the distal surface along a first axis, the passage configured to accommodate the handheld ultrasound probe, wherein the plate-shaped member has a thickness between the proximal surface and the distal surface along the first axis and a width along a second axis perpendicular to the first axis, the thickness being less than the width, and when the handheld ultrasound probe is inserted in the attachment assembly, a portion of a transducer head of the handheld ultrasound probe protrudes from the distal surface of the plate-shaped member.

In general, one or more embodiments of the disclosure relate to an attachment assembly for ergonomic handling of a handheld ultrasound probe, the attachment assembly comprising: a shell that attaches to the handheld ultrasound probe and partially covers the handheld ultrasound probe; and strap anchors that are each connected to the shell and are disposed at opposite ends of the shell.

In general, one or more embodiments of the disclosure relate to an attachment for ergonomic handling of a handheld ultrasound probe, the attachment comprising: a curved bar that is continuous between a proximal attachment flange, of the curved bar, that attaches to a first location of the handheld ultrasound probe and a distal attachment flange, of the curved bar, that attaches to a second location of the handheld ultrasound probe, wherein a segment of the curved bar forms a bar handle.

Other aspects of the disclosure will be apparent from the following description and the appended claims.

### BRIEF DESCRIPTION OF DRAWINGS

FIG. 1 shows an attachment assembly attached to a handheld ultrasound probe, according to one or more embodiments.

FIG. 2A shows a three-dimensional rendering of an attachment assembly attached to a handheld ultrasound probe, as viewed from a side angle, according to one or more embodiments.

FIG. 2B shows a three-dimensional rendering of an attachment assembly attached to a handheld ultrasound probe, as viewed from an oblique angle, according to one or more embodiments.

FIG. 2C shows a three-dimensional, partially transparent rendering of an attachment assembly attached to a handheld ultrasound probe, as viewed from an oblique angle, according to one or more embodiments.

FIG. 3A shows a schematic of an attachment assembly attached to a handheld ultrasound probe, as viewed from a side angle, according to one or more embodiments.

FIG. 3B shows a schematic of an attachment assembly attached to a handheld ultrasound probe, as viewed along the length of the handheld ultrasound probe, according to one or more embodiments.

FIG. 4A shows a three-dimensional rendering of an attachment assembly for a handheld ultrasound probe, as viewed from an oblique angle, according to one or more embodiments.

FIG. 4B shows a three-dimensional rendering of an attachment assembly attached to a handheld ultrasound probe, as viewed from an oblique angle, according to one or more embodiments.

FIG. 5A shows a schematic of an attachment assembly attached to a handheld ultrasound probe, as viewed from a side angle, according to one or more embodiments.

FIG. 5B shows a schematic of an attachment assembly attached to a handheld ultrasound probe, as viewed from a side angle, according to one or more embodiments.

FIG. 6 shows a three-dimensional rendering of an attachment for a handheld ultrasound probe, as viewed from an oblique angle, according to one or more embodiments.

FIG. 7A shows a three-dimensional rendering of an attachment attached to a handheld ultrasound probe, as viewed from an oblique angle, according to one or more embodiments.

FIG. 7B shows a three-dimensional rendering of an attachment attached to a handheld ultrasound probe, as viewed from a side angle, according to one or more embodiments.

FIG. 7C shows a three-dimensional rendering of an attachment attached to a handheld ultrasound probe, as viewed from a side angle, according to one or more embodiments.

FIG. 8A shows a schematic of an attachment attached to a handheld ultrasound probe, as viewed from a side angle, according to one or more embodiments.

FIG. 8B shows a schematic of an attachment attached to a handheld ultrasound probe, as viewed along the length of the handheld ultrasound probe, according to one or more embodiments.

FIG. 8C shows a schematic of an attachment attached to a handheld ultrasound probe, as viewed along the length of the handheld ultrasound probe, according to one or more embodiments.

FIG. 9 shows a block diagram of an example of an ultrasound device according to one or more embodiments.

FIG. 10 shows a schematic block diagram of an example ultrasound system, according to one or more embodiments.

FIG. 11 shows an example handheld ultrasound probe, according to one or more embodiments.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

Throughout the application, ordinal numbers (e.g., first, second, third) may be used as an adjective for an element (i.e., any noun in the application). The use of ordinal numbers is not to imply or create a particular ordering of the elements nor to limit any element to being only a single element unless expressly disclosed, such as by the use of the terms “before,” “after,” “single,” and other such terminology. Rather the use of ordinal numbers is to distinguish between the elements. By way of an example, a first element is distinct from a second element, and the first element may encompass more than one element and may succeed (or precede) the second element in an ordering of elements.

In general, embodiments of the disclosure provide attachments or attachment assemblies for ergonomic handling of a handheld ultrasound probe. One or more embodiments provides an attachment assembly that has a plate-shaped member that may allow for improved handling and manipulation of a handheld ultrasound probe. The plate-shaped member has a passthrough that accommodates the handheld ultrasound probe. The attachment assembly, including the plate-shaped member, may be divided into assembly sections such that the attachment assembly can be assembled around the handheld ultrasound probe and held together by fasteners. Other embodiments provide an attachment assembly that includes a shell that attaches to, and partially covers, the body of the handheld ultrasound probe. This attachment assembly may include strap anchors that allow a flexible strap to be attached to the attachment assembly, such that a user's hand may be inserted between the strap and the shell in order to allow the user to comfortably hold the handheld ultrasound probe and reduce strain and fatigue. Still other embodiments provide an attachment that includes a curved bar that attaches to a handheld ultrasound probe at two attachment points on the body of the handheld ultrasound probe. The curved bar extends away from the handheld ultrasound probe at the attachment points and curves to form a continuous curved bar between the two attachment points. A segment of the curved bar may be used as a bar handle for gripping and manipulating the handheld ultrasound probe. The attachment may also include a palm handle that is disposed near an end of the handheld ultrasound probe such that a user may comfortably push the handheld ultrasound probe against a body or medium being imaged using a palm of a hand.

The described configurations of attachments and attachment assemblies for handheld ultrasound probes, in accordance with embodiments of the disclosure, therefore allow for ergonomic handling and manipulation of the handheld ultrasound probe and for reduced strain and fatigue in a user or technician operating the handheld ultrasound probe.

### First Set of Embodiments

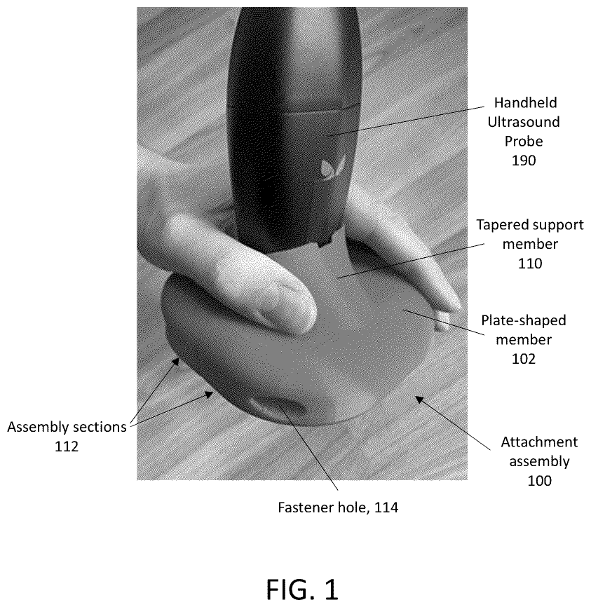

FIG. 1 shows an attachment assembly 100 attached to a handheld ultrasound probe 190, according to one or more embodiments. The attachment assembly 100 comprises a plate-shaped member 102, that is a generally flat part of the attachment. As shown in FIG. 1, the plate-shaped member 102 may be used as an ergonomic grip or handle for holding and/or manipulation by a human hand. In this way, the attachment assembly 100 may provide an improved grip and/or an improved ability for a user to manipulate the handheld ultrasound probe 190, which may also help suppress fatigue caused by prolonged operation of the handheld ultrasound probe 190. Additionally, the improved grip and ability to manipulate the handheld ultrasound probe 190 may also allow for patients to more easily perform ultrasound imaging on themselves, especially in cases where the patient is elderly, arthritic, obese, or disabled.

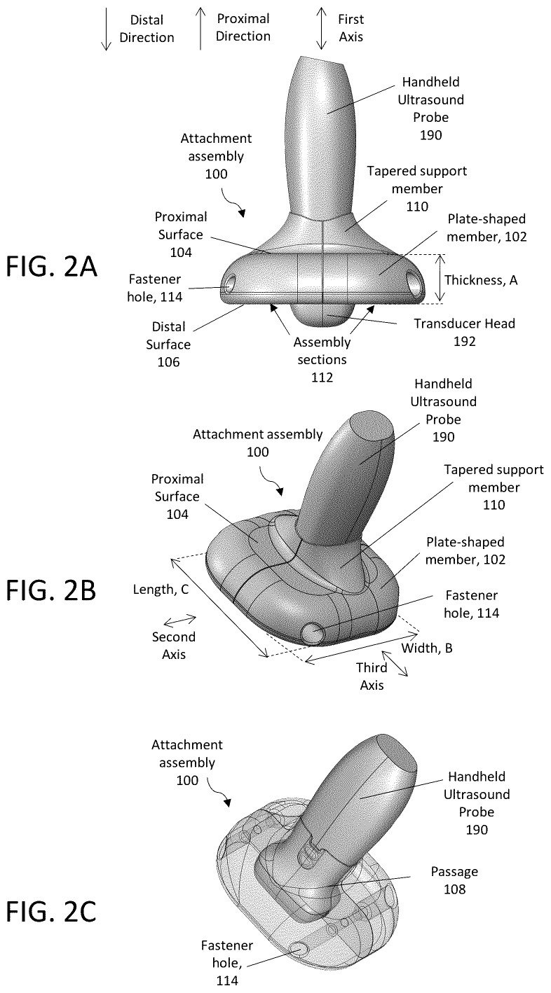

FIGS. 2A-C show three-dimensional renderings of an attachment assembly 100 attached to a handheld ultrasound probe 190, as viewed from multiple angles, according to one or more embodiments. As shown in FIG. 2A, the plate-shaped member 102 has a proximal surface 104 and a distal surface 106. As shown in FIG. 2C, where the plate-shaped member 102 is shown as partially transparent, the plate-shaped member 102 comprises a passage 108 between the proximal surface 104 and the distal surface 106, along a first axis. The passage 108 is an opening that is configured to accommodate the handheld ultrasound probe 190. In other words, the passage 108 is an open space through the plate-shaped member 102 with an opening at both the proximal surface 104 and the distal surface 106. The handheld ultrasound probe 190 spans the passage 108, when the attachment assembly 100 is attached to the handheld ultrasound probe 190. The direction through which the handheld ultrasound probe 190 spans the passage 108 defines the first axis (i.e., along the length of the handheld ultrasound probe 190). In some embodiments, the passage 108 is disposed so as to be centered along the first axis (i.e., in the center of the plate-shaped member 102. However, in other embodiments, the passage 108 may be offset in any direction from the first axis such that the passage 108 is not centered within the plate-shaped member 102.

The distal surface 106 of the plate-shaped member 102 is disposed on the side of the plate-shaped member 102 that is closest to the transducer head of the handheld ultrasound probe 190 (i.e., the part of the handheld ultrasound probe 190 that includes the ultrasound transducers used for transmitting and receiving ultrasound signals). The proximal surface 104 is opposite to the distal surface 106, in a direction along the first axis. In some embodiments, the distal surface 106 is substantially flat, as shown in FIG. 2A. However, in other embodiments the outer edge of the distal surface 106 may be wholly or partially beveled, chamfered, or filleted such that a portion of the distal surface 106 is curved. In still other embodiments, the entirety of the distal surface 106 may be curved. Similarly, in some embodiments, the outer edge of the proximal surface 104 may be beveled, chamfered or filleted, as shown in FIG. 2A, such that a portion of the proximal surface 104 is curved. However, in other embodiments, the proximal surface 104 may also be substantially flat. In still other embodiments, the entirety of the proximal surface 104 may be curved.

In one or more embodiments, when the handheld ultrasound probe 190 is inserted in the attachment assembly 100, a portion of the transducer head 192 of the handheld ultrasound probe 190 protrudes from the distal surface 106 of the plate-shaped member 102. As shown in the FIG. 2A, a portion the transducer head 192 of the handheld ultrasound probe 190 remains external to the attachment assembly 100. In some embodiments, only a small portion of the transducer head 192 protrudes from the distal surface 106 of the plate-shaped member 102, as shown in FIG. 2A. In other embodiments, larger portions of the transducer head 192, or the entire transducer head 192 may protrude from the distal surface 106 of the plate-shaped member 102. Because the transducer head 192 protrudes from the attachment assembly 100 in this way, a user may be able to use the attachment assembly 100 to comfortably and effectively push the transducer head 192 of the handheld ultrasound probe 190 against a body or medium that is being imaged.

The plate-shaped member 102 has a thickness A between the distal surface 106 and the proximal surface 104, along the first axis. The plate-shaped member 102 also has a width B, along a second axis that is perpendicular to the first axis. In one or more embodiments, the thickness A of the plate-shaped member 102 is less than the width B of the plate-shaped member 102. In some embodiments, the thickness A of the plate-shaped member 102 may be less than one third of the width B of the plate-shaped member 102.

The plate-shaped member 102 also has a length C, along a third axis that is perpendicular to both the first axis and the second axis. In some embodiments, the length C of the plate-shaped member 102 is equal to the width B of the plate-shaped member 102. In other embodiments, the length C may be either longer or shorter than the width B. In some embodiments, when viewed from along the first axis, the shape of the plate-shaped member 102 may be substantially square, with rounded corners, as shown in the FIG. 2B. However, the shape of the plate-shaped member 102, when viewed from along the first axis, is not limited to the shape shown in the figures. In other embodiments, the shape of the plate-shaped member 102, when viewed from along the first axis may be either square or rectangular with either rounded or sharp corners. In still other embodiments, the shape of the plate-shaped member 102, when viewed from along the first axis, may be substantially the shape of a circle, oval, triangle, polygon, or any other suitable shape.

In one or more embodiments, the attachment assembly 100 may also comprise a tapered support member 110 that is rigidly connected to the plate-shaped member 102. The tapered support member 110 connects directly to the proximal surface 104 of the plate-shaped member 102. At the connection between the tapered support member 110 and the proximal surface 104 of the plate-shaped member 102, the tapered support member 110 has its greatest length C and/or width B. The tapered support member 110 tapers such that the length C and/or width B decreases in the proximal direction. The tapered support member 110 also has a passage that passes through the tapered support member 110 and is an extension of the passage 108. The passage through the tapered support member 110 is also configured to accommodate the handheld ultrasound probe 190. The passage through the tapered support member 110 accommodates a segment of the handheld ultrasound probe 190 that is adjacent to the segment of the handheld ultrasound probe 190 that is accommodated by the passage 108. When the attachment assembly 100 is attached to the handheld ultrasound probe 190, the tapered support member 110 provides support and stabilization to the plate-shaped member 102, aiding in securing and immobilizing the plate-shaped member 102 with respect to the handheld ultrasound probe 190.

In one or more embodiments, the attachment assembly 100 may be divided into two assembly sections 112 along a plane that is defined by the first axis and the second axis. In other words, the assembly sections 112 are sections of the attachment assembly 100 that may be assembled around the handheld ultrasound probe 190. In this way, the attachment assembly 100 may be formed such that the passage 108 tightly accommodates irregularly shaped ultrasound probes since the ultrasound probe does not have to be inserted through the passage 108. In some embodiments, the attachment assembly 100 is divided symmetrically such that each of the assembly sections 112 are similar size and shape. However, in other embodiments, the attachment assembly 100 may be divided asymmetrically such that each of the assembly sections 112 are different sizes and potentially different shapes. For example, in the case where the passage 108 is not centered within the plate-shaped member 102, as discussed previously, the two assembly sections 112 may be different sizes.

In one or more embodiments, the assembly sections 112 are fastened together by fasteners. In some embodiments, the assembly sections 112 each have one or more fastener holes 114. The fastener holes 114 may extend fully through the assembly sections 112 and mate with corresponding fastener holes 114 in the other assembly section 112. Fasteners may be inserted into the fastener holes 114 in order to hold the assembly sections 112 together when the attachment assembly 100 is attached to handheld ultrasound probe 190. In some embodiments, the fastener holes 114 may have an internal section that is narrower than the rest of the fastener hole 114, as shown in FIG. 2C. In this case, the fasteners may be bolts that are inserted into the fastener hole 114 of one assembly section 112, through the narrow segment of the fastener hole 114, and through the narrow segment of the mating fastener hole 114 in the other assembly section 112. A nut may be inserted into the fastener hole 114 of the other assembly section 112 and affixed to the bolt, so that the bolt and nut may apply a force to the narrow segments of the fastener holes 114, resulting in the assembly sections 112 of the attachment assembly 100 being held in contact with each other. In this way, the bolts and nuts inserted into the fastener holes 114 can hold the attachment assembly 100 in place on the handheld ultrasound probe 190.

In other embodiments, the fasteners may be screws. In this case the fastener holes 114 may fully extend through one of the assembly sections 112, while the fastener holes 114 in the opposing assembly section 112 are threaded and do not extend fully through the opposing assembly section 112. In this way, when the assembly sections 112 are joined together, such that corresponding fastener holes 114 are mated together, a screw may be inserted into the open fastener hole 114 and then screwed into the mating threaded fastener hole 114. In this way the threads of the screw apply a force to the threads of the mating fastener hole 114 holding the assembly sections 112 of the attachment assembly 100 together, thereby securing the attachment assembly 100 to the handheld ultrasound probe 190.

In other embodiments, other configurations of fasteners and fastening mechanisms may also be utilized. Examples of other possible fasteners that may be used to hold the assembly sections 112 together include, but are not limited to: pins, nails, rivets, clasps, latches, springs, hooks, straps, or any other suitable mechanism for attaching and holding the assembly sections 112 together.

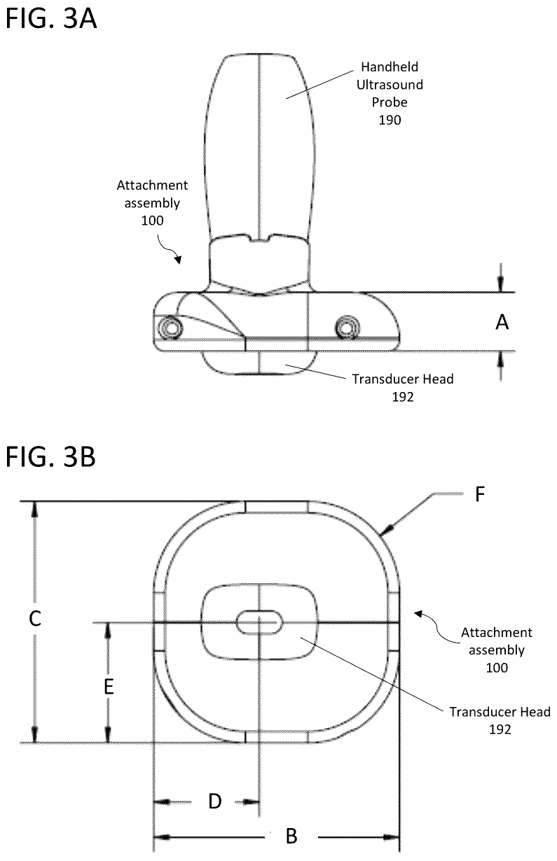

In one or more embodiments, the attachment assembly 100 may have the dimensions A-F specified in Table 1, as shown in FIGS. 3A and 3B.

TABLE 1

| Label | Value | |
|---|---|---|
| A | 26₋₁₅⁺²⁵ | mm |
| B | 107 ± 50 | mm |
| C | 106 ± 50 | mm |
| D | 46₋₁₅⁺²⁵ | mm |
| E | 53 ± 20 | mm |

| | |
|---|---|
| F | Radius of 40 ± 20 mm |

However, the attachment assembly 100 is not limited to only the dimensions listed in Table 1. Any of the dimensions shown FIGS. 3A and 3B may have different values than those specified in Table 1.

The attachment assembly 100 may be made of any suitable material including, but not limited to, hard plastics, soft plastics, rubbers, silicones, woods, metals or any other material, composite material, or combination of materials.

### Second Set of Embodiments

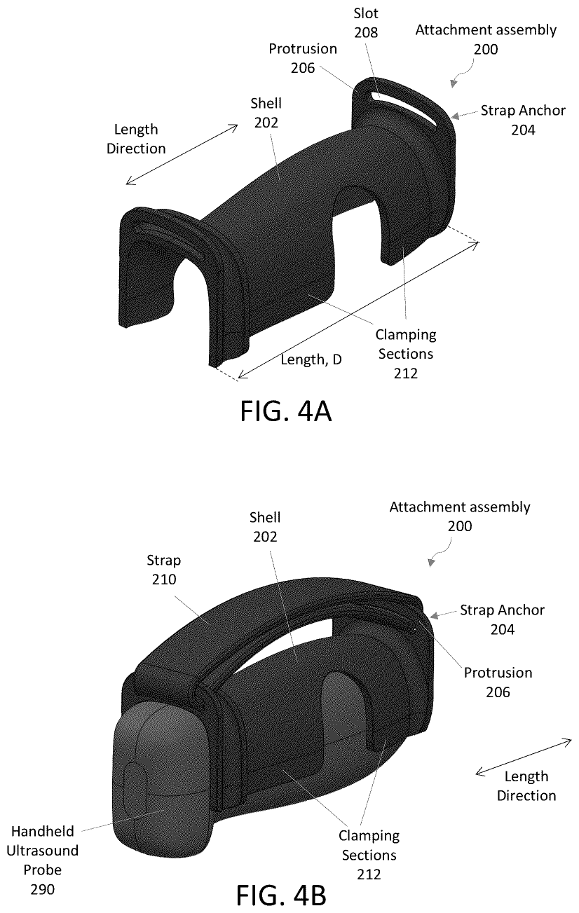

FIGS. 4A-4B show three-dimensional renderings of an attachment assembly 200 for a handheld ultrasound probe, as viewed from an oblique angle, according to one or more embodiments. As shown in FIG. 4A, the attachment assembly 200 includes a shell 202 that attaches to the handheld ultrasound probe and partially covers the handheld ultrasound probe (not shown). The attachment assembly 200 also includes strap anchors 204 that are each connected to the shell 202. The shell 202 may be made of any suitable material including, but not limited to, hard plastics, soft plastics, rubbers, silicones, fabrics, metals or any other material, composite material, or combination of materials that can be made to partially cover and attach to the handheld ultrasound probe. Similarly, the strap anchors 204 may be made of any suitable material including, but not limited to, hard plastics, soft plastics, rubbers, silicones, fabrics, metals or any other materials, composite material or combination of materials that can be connected to the shell 202. In some embodiments, the strap anchors 204 may be made of the same material as the shell 202, while in other embodiments, the strap anchors 204 may be made of different materials than the materials of the shell 202.

In some embodiments, the strap anchors 204 may be disposed at opposite ends of the shell 202 in a length direction, as shown in FIG. 4A. The length direction is defined by the distance between the strap anchors, or as the direction along the length of the handheld ultrasound probe when the attachment assembly 200 is attached to the handheld ultrasound probe. In other embodiments, the strap anchors 204 may not be disposed at opposite ends of the shell 202 in the length direction. For example, one or both of the strap anchors 204 may also be disposed to be between the ends of the shell 202 in the length direction or the strap anchors 204 may also be disposed at any other suitable locations on the shell 202.

In one or more embodiments, the strap anchors 204 may be protrusions 206 that extend away from the shell 202. In some embodiments, the protrusions 206 extend away from the shell 202 in a direction that is substantially perpendicular to the length direction. However, in other embodiments, the protrusions 206 may extend in other directions. For example, the protrusions 206 may extend at an angle that is oblique with respect to the length direction. Alternatively, the protrusions 206 may be bent or curved such that the protrusions 206 extend away from the shell 202 in a direction perpendicular to the length direction and then bend or curve to be substantially parallel with the length direction. In some embodiments, the protrusions 206 (i.e., the strap anchors 204) may each have a slot 208. The slot 208 may be a long and narrow hole or opening in the protrusion 206, where the longest dimension of the hole is substantially oriented in a direction that is perpendicular to both the direction of extension of the protrusions 206 and perpendicular to the length direction. The slot 208 may also be either straight or curved.

However, the strap anchors 204 should not be limited by the above description. The strap anchors 204 may be any other structure that allows a strap, rope, or ribbon to be attached to the shell 202 of the attachment assembly 200. For example, the strap anchors 204 may be loops of various shapes, that may be moveable or not moveable, attached to the shell 202. Alternatively, the strap anchors 204 may be brackets that are attached to the shell 202 such that a space exists between part of the bracket and the shell 202, allowing the strap to be fed through the space. In some embodiments, a strap may be directly attached to the shell 202 via fasteners such as screws, rivets, staples, stitching, or any other suitable fastener. In this case the fasteners may be considered to be the strap anchors 204.

In one or more embodiments, the attachment assembly 200 further comprises a flexible strap 210 that is attached to each of the strap anchors 204. The strap 210 may be made of any flexible material. For example, the strap 210 may be made out of woven or non-woven fabrics that are based on natural or synthetic materials. Additionally, the strap 210 may be made of flexible soft or hard plastic materials, silicones, or rubber. In one or more embodiments, the strap 210 may feed through the strap anchors 204 (i.e., the slots 208), and the strap 210 may attach to itself in order to form a loop around the strap anchor 204. In some embodiments, the strap 210 may attach to itself via hook and loop connectors, clips, clasps, buckles, rivets, cam buckles, stitching, or any other suitable attachment between a strap and itself. By having the strap attached to the strap anchors, an opening may be formed between the strap 210 and the shell 202 such that a user's hand may be inserted between the strap 210 and the shell 202 so that the user may be able to grip and control the handheld ultrasound probe in a way that requires less strength and dexterity than unassisted gripping of a handheld ultrasound probe. In this way, an operator using the handheld ultrasound probe may experience less strain or fatigue when using the handheld ultrasound probe. Additionally, a patient that is performing an ultrasound self-scan may be able to more easily image difficult to reach locations, especially in the case of patients who are elderly, arthritic, obese, or disabled.

In one or more embodiments, the shell 202 includes one or more clamping sections 212 with a substantially C-shaped opening, where the clamping section 212 partially accommodates the handheld ultrasound probe in the C-shaped opening. In other words, the clamping sections 212 allow the shell 202 to be attached to the handheld ultrasound probe 290, as shown in FIG. 4B. The C-shape of the clamping section 212 allows the clamping section 212 to retain a portion of the handheld ultrasound probe 290 within the hollow interior of the C-shape. In one or more embodiments, the C-shaped opening accommodates greater than 50% of the cross-sectional area of the handheld ultrasound probe 290, at the part of the handheld ultrasound probe 290 to which the clamping section 212 is attached. In other words, the ends of the C-shape extend past the widest part of the body of the handheld ultrasound probe 290, and therefore, partially wrap around the body of the handheld ultrasound probe 290 so that the handheld ultrasound probe 290 is unable to inadvertently fall out of the clamping section 212.

In one or more embodiments, the attachment assembly 200 may have two clamping sections 212, as shown in FIGS. 4A and 4B. However, in other embodiments, the attachment assembly 200 may have only one clamping section 212, or three or more clamping sections 212.

In one or more embodiments, the clamping sections 212 may be made of a hard-elastic material, and the C-shaped opening may widen when the handheld ultrasound probe 290 is inserted into the attachment assembly 200. In other words, the clamping sections 212 may be flexible in that the C-shaped opening may be expanded. However, the clamping sections 212 may also be elastic in that the clamping sections 212 may tend to return to the original size and shape of the C-shaped opening when not being forced to expand. In this way, the clamping sections 212 allow for the body of the handheld ultrasound probe 290 to be inserted through an opening that is smaller than the widest part of the body of the handheld ultrasound probe 290, when inserting or removing the handheld ultrasound probe 290, by forcing the opening to widen. However, once the handheld ultrasound probe 290 is fully inserted to the attachment assembly 200, the clamping sections 212 contract back toward their original size and shape such that the body of the handheld ultrasound probe 290 is retained by the clamping section 212. Examples of hard-elastic materials may include any polymer-based plastic or any other material that has elastic properties such that it is flexible, but tends to return to its original size and shape.

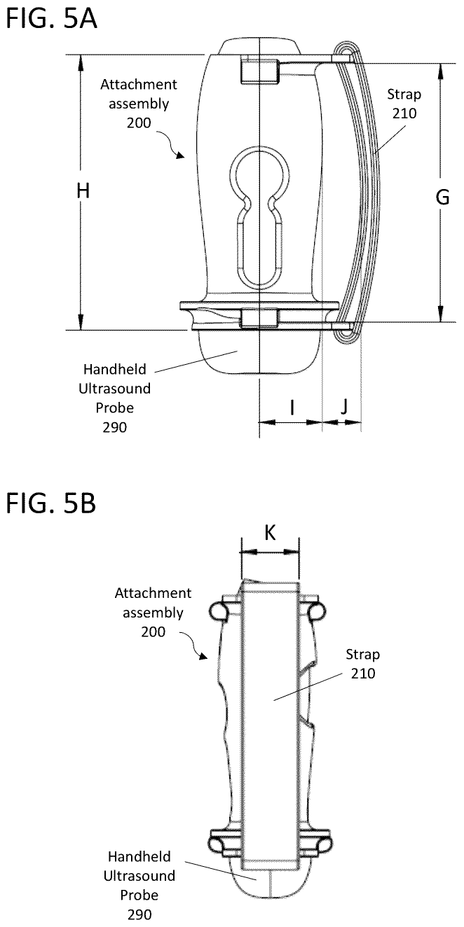

In one or more embodiments, the attachment assembly 200 may have the dimensions G-K specified in Table 2, as shown in FIGS. 5A and 5B.

TABLE 2

| Label | Value | |
|---|---|---|
| G | 108 ± 50 | mm |
| H | 115 ± 50 | mm |
| I | 27₋₁₅⁺²⁵ | mm |
| J | 16₋₁₀⁺²⁰ | mm |
| K | 25₋₁₅⁺⁵⁰ | mm |

However, the attachment assembly 200 is not limited to only the dimensions listed in Table 2. Each of the dimensions shown FIGS. 5A and 5B may have different values than those specified in Table 2.

### Third Set of Embodiments

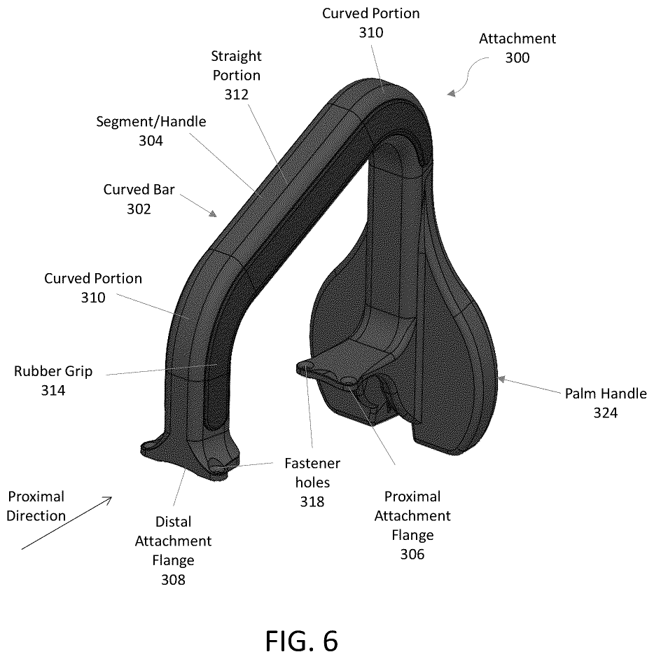

FIG. 6 shows a three-dimensional rendering of an attachment 300 for a handheld ultrasound probe, as viewed from an oblique angle, according to one or more embodiments. As shown in FIG. 6, the attachment 300 includes a curved bar 302 that is continuous between a proximal attachment flange 306, of the curved bar 302, and a distal attachment flange 308, of the curved bar 302. The proximal attachment flange 306 attaches to a first location of the handheld ultrasound probe (not shown) and the distal attachment flange 308 attaches to a second location of the handheld ultrasound probe. A segment 304 of this curved bar 302 forms a bar handle that a user can grip in order to achieve an improved grip and ergonomic handling of the handheld ultrasound probe.

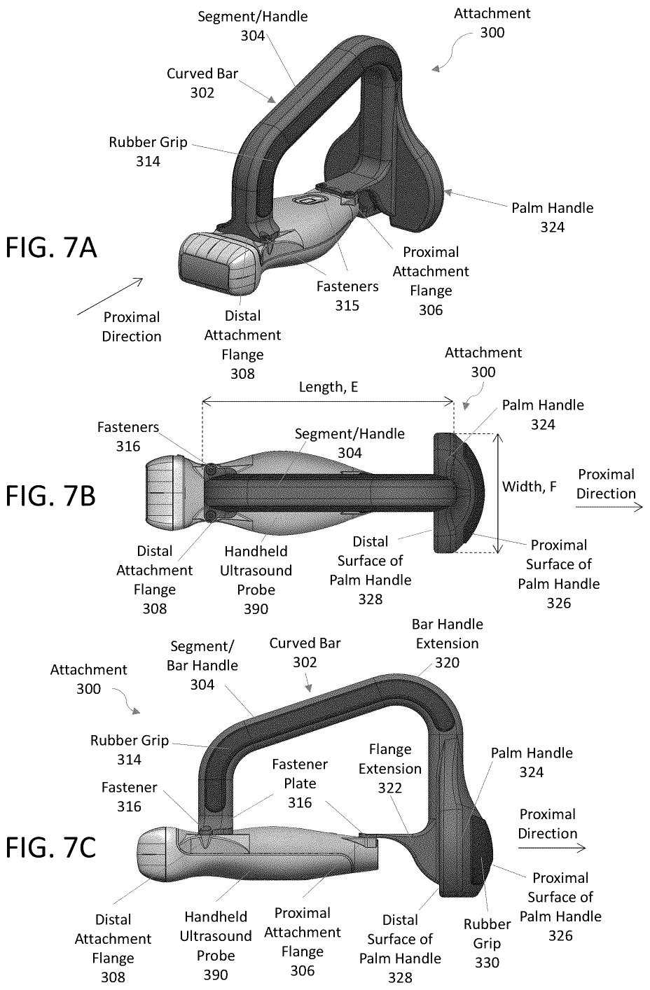

In some embodiments, the curved bar 302 may have two or more curved portions 310 separated by a straight portion 312. The curved portions 310 may each curve by the same angle or by different angles, as shown in FIG. 6. In the case where the curved sections curve by the same angle, the straight portion 312 may be substantially parallel to a proximal direction. (As shown in FIG. 6 and FIG. 7A, the proximal direction is defined as the direction from the distal attachment flange 308 to the proximal attachment flange 306, and this direction is along a long axis of the handheld ultrasound probe when the attachment 300 is attached to the handheld ultrasound probe.) On the other hand, in the case where the curved sections curve by different angles, the straight section may slope, in the proximal direction, away from or toward the handheld ultrasound probe, when the attachment 300 is attached to the handheld ultrasound probe. In other embodiments, the curved bar 302 may continuously curve, from the proximal attachment flange 306 to the distal attachment flange 308, in a loop shape.

In one or more embodiments, the segment 304 that forms the bar handle may be spatially separated from the handheld ultrasound probe. The spatial separation between the handheld ultrasound probe and the segment 304 establishes an opening between the bar handle and the handheld ultrasound probe that allows room for a users' hand or fingers to be positioned between the bar handle and the handheld ultrasound probe while the user is gripping the attachment 300. In other words, the segment 304 of the curved bar 302 that forms the bar handle may be offset from the handheld ultrasound probe in a direction that is perpendicular to the proximal direction, allowing the user to grip the bar handle and ergonomically manipulate the handheld ultrasound probe in order to facilitate patient self-scanning for ultrasound imaging. In particular, the handle may enable patients to self-scan otherwise inaccessible locations of the body, especially in the case of elderly, arthritic, obese, or disabled patients.

In one or more embodiments, the curved bar 302 may include a rubber grip 314. In some embodiments, the rubber grip 314 may include one or more strips of a rubber or silicone material, along the length of the curved bar 302, that partially cover the bar handle. In other embodiments, the rubber grip 314 may wrap fully around the curved bar 302 in order to fully cover at least a segment 304 of the bar handle. The rubber grip 314 may serve as an anti-slip surface that improves a user's grip of the bar handle.

FIGS. 7A-7C shows the attachment 300 as seen from multiple angles, when the attachment 300 is attached to the handheld ultrasound probe 390. As shown in FIGS. 7A-7C, the proximal attachment flange 306 and the distal attachment flange 308 are removably attachable to the handheld ultrasound probe 390. In one or more embodiments, the proximal attachment flange 306 and the distal attachment flange 308 may be removably attachable to the handheld ultrasound probe 390 by fasteners 315. In one or more embodiments, the fasteners 315 may be screws. In these embodiments, the handheld ultrasound probe 390 to which the attachment 300 is attached may also have threaded holes that are configured to be mated with the proximal attachment flange 306 and the distal attachment flange 308. In other embodiments, any other suitable fasteners may also be used to attach the attachment 300 to the handheld ultrasound probe 390. For example, holes may extend through the body of the handheld ultrasound probe 390 such that bolts can be used to attach the proximal attachment flange 306 and the distal attachment flange 308 to the handheld ultrasound probe 390. Various other fasteners may also be used to attach the proximal attachment flange 306 and the distal attachment flange 308 to the handheld ultrasound probe 390. These include, but are not limited to, clips, rivets, springs, and hooks, or any other suitable fasteners.

In one or more embodiments, the proximal attachment flange 306 and the distal attachment flange 308 each comprise a fastener plate 316, that has one or more fastener holes 318, and the fasteners 315 are removably inserted into the fastener holes 318. The fastener plate 316 is a flat surface against which the head of a screw, bolt, or other fastener can apply force in order to firmly hold the attachment 300 in place when attached to the handheld ultrasound probe 390. In some embodiments, as shown in FIGS. 7A-7C, the fastener plate 316, extends away from the curved bar 302 such that the fasteners holes are offset from the curved bar 302 in a direction that is perpendicular to the proximal direction. In other embodiments, for example, in a case where the attachment flanges may need to be smaller in size, the proximal attachment flange 306 and/or the distal attachment flange 308 may be disposed internal to the ends of the curved bar 302, such that the fastener holes 318 extend through the curved bar 302, allowing access for inserting the fasteners 315.

However, in some embodiments, the attachment 300 may attach to a handheld ultrasound probe using other mechanisms. For example, the curved bar 302 may attach directly to a shell that accommodates and attaches to a handheld ultrasound probe in a similar manner as the attachment assembly 200, as described above. Alternatively, the curved bar 302 may attach directly to a sleeve that has a passage (e.g., analogous to the passage 108 of the previously described embodiment) that accommodates a portion of the body of a handheld ultrasound probe. The sleeve may be made of a rigid material and have multiple sections that clamp around the body of the handheld ultrasound probe, or the sleeve may be made of a flexible material that slides onto a handheld ultrasound probe. In these embodiments, the attachment 300 may be attached directly to any suitable handheld ultrasound probe without the need for the body of the handheld ultrasound probe to include special parts for accommodating fasteners such as, for example, threaded holes.

In one or more embodiments, as shown in FIG. 7C, a portion of the segment 304 that forms the bar handle extends past the proximal attachment flange 306, in the proximal direction. In other words, a bar handle extension 320 of the curved bar 302 may extend past the proximal attachment flange 306 in the proximal direction. The bar handle extension 320 allows the segment 304 that forms the bar handle to be longer. In this way, the bar handle may be larger to allow for more control in handling or manipulating the handheld ultrasound probe 390. Additionally, the larger bar handle may better accommodate larger hand sizes. Because the bar handle extension 320 extends past the proximal attachment flange 306, in the proximal direction, a flange extension 322, of the curved bar 302, also extends, away from the proximal attachment flange 306, in the proximal direction in order complete the curved bar 302 so that it continuously extends from the distal attachment flange 308 to the proximal attachment flange 306.

In one or more embodiments, a palm handle 324 is attached to the curved bar 302 and is offset, in the proximal direction, from the proximal attachment flange 306. In other words, in the case where the bar handle extension 320 extends past the proximal attachment flange 306 in the proximal direction, some embodiments may include a palm handle 324. The palm handle 324 may be disposed at the proximal end of the handheld ultrasound probe 390 when the attachment 300 is attached to the handheld ultrasound probe 390, or the palm handle 324 may be offset in the proximal direction from the proximal end of the handheld ultrasound probe 390.

The palm handle 324 may extend away from the curved bar 302 in at least one direction that is perpendicular to the proximal direction. In some embodiments, the palm handle 324 may extend in multiple directions that are perpendicular to the proximal direction such that the palm handle 324 has a large surface area suitable for pushing by the palm of a user's hand. In this way, because the palm handle 324 is substantially aligned along the long axis of the handheld ultrasound probe 390 when the attachment 300 is attached to the handheld ultrasound probe 390, it may allow a user to comfortable apply a force to the handheld ultrasound probe 390, effectively pushing the transducer head of the probe into the body or medium that is being imaged. In some embodiments, the palm handle 324 is substantially teardrop-shaped when viewed from the proximal direction (i.e., when viewed from along the long axis of the handheld ultrasound probe 390 when the attachment 300 is attached to the handheld ultrasound probe 390). However, in other embodiments, the palm handle 324 may be other shapes, when viewed from the proximal direction, including but not limited to circular/disk-shaped, square-shaped, rectangular-shaped, triangular-shaped, hexagonal-shaped, octagonal-shaped, or any other suitable shape.

In some embodiments, as shown in FIG. 7B, the palm handle 324 may extend in the direction that is perpendicular to the proximal direction to be greater than or equal to three times the width of the bar handle, in the same direction. In other embodiments, the palm handle 324 may be any other suitable size that allows the user to push against the palm handle 324 with the palm of a hand.

The palm handle 324 also has a proximal surface 326 that faces substantially away from the handheld ultrasound probe 390. The proximal surface 326 is opposite to a distal surface 328 that faces toward the handheld ultrasound probe 390. In other words, the distal surface 328 of the palm handle 324 faces substantially opposite to the proximal direction, while the proximal surface 326 faces substantially toward the proximal direction. In one or more embodiments, the proximal surface 326 is curved so as to be substantially convex. In other words, the proximal surface 326 curves such that it bulges toward the proximal direction. This convex shape may allow the palm handle 324 to be comfortably pushed against by a user with the palm of a hand. However, in other embodiments, the proximal surface 326 of palm handle 324 may instead be substantially flat or substantially concave.

In one or more embodiments, the palm handle 324 may include a rubber grip 330 that is attached to the proximal surface 326 of the palm handle 324. In some embodiments, the rubber grip 330 may include one or more areas of rubber or silicone material attached to the palm handle 324. The rubber grip 330 may partially cover the proximal surface 326 of the palm handle 324, or the rubber grip 330 may fully cover the proximal surface 326 of the palm handle 324. The rubber grip 330 may serve as an anti-slip surface that improves a user's grip of the palm handle 324 and reduces the likelihood that the user's palm slips off the palm handle 324 of the attachment 300.

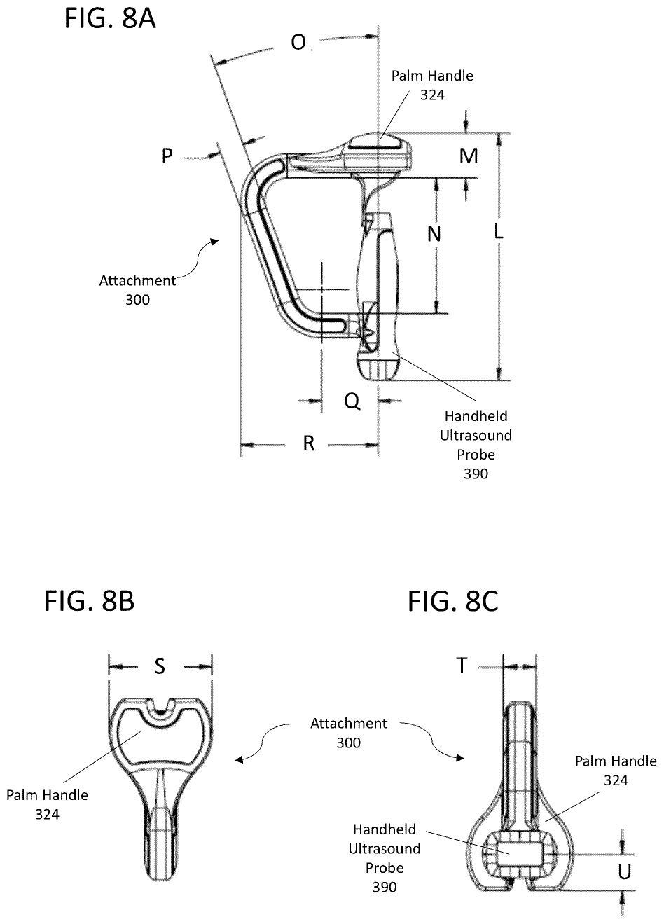

In one or more embodiments, the attachment assembly 200 may have the dimensions L-U specified in Table 3, as shown in FIGS. 8A-8C.

TABLE 3

| Label | Value | |
|---|---|---|
| L | 206₋₇₅⁺⁵⁰ | mm |
| M | 37 ± 20 | mm |
| N | 113 ± 50 | mm |
| O | Angle of 20°_{-20°}^{+20°} | |
| P | 20 ± 10 | mm |
| Q | 47 ± 20 | mm |
| R | 114₋₇₅⁺²⁵ | mm |
| S | 80 ± 25 | mm |
| T | 25 ± 10 | mm |
| U | 27₋₁₀⁺²⁰ | mm |

However, the attachment 300 is not limited to only the dimensions listed in Table 3. Each of the dimensions shown FIGS. 8A-8C may have different values than those specified in Table 3.

The attachment 300 may be made of any suitable material including, but not limited to, hard plastics, soft plastics, rubbers, silicones, woods, metals or any other material, composite material, or combination of materials.

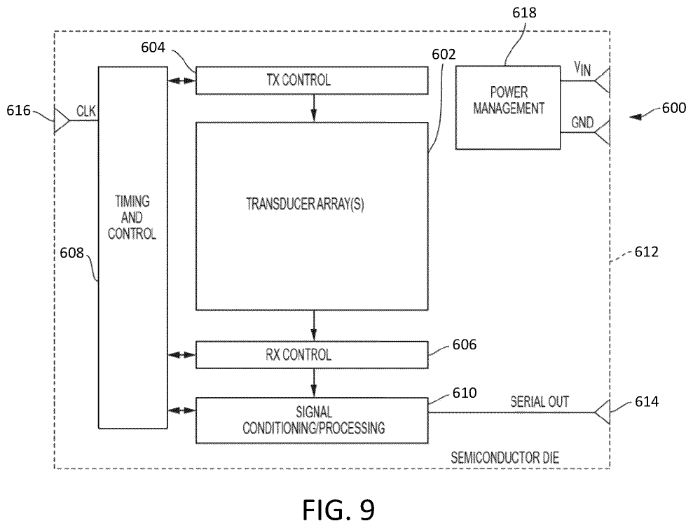

FIG. 9 is a block diagram of an example of an ultrasound device in accordance with some embodiments of the technology described herein. The illustrated ultrasound device may, in some embodiments, be part of the handheld ultrasound probe, 190, 290, and/or 390. The illustrated ultrasound device may implement the signal processing techniques described herein, including the coherence imaging techniques described herein. The illustrated ultrasound device 600 may include one or more ultrasonic transducer arrangements (e.g., arrays) 602, transmit (TX) circuitry 604, receive (RX) circuitry 606, a timing and control circuit 608, a signal conditioning/processing circuit 610, and/or a power management circuit 618.

The one or more ultrasonic transducer arrays 602 may take on any of numerous forms, and aspects of the present technology do not necessarily require the use of any particular type or arrangement of ultrasonic transducer cells or ultrasonic transducer elements. For example, multiple ultrasonic transducer elements in the ultrasonic transducer array 602 may be arranged in one-dimension, or two-dimensions. Although the term “array” is used in this description, it should be appreciated that in some embodiments the ultrasonic transducer elements may be organized in a non-array fashion. In various embodiments, each of the ultrasonic transducer elements in the array 602 may, for example, include one or more capacitive micromachined ultrasonic transducers (CMUTs), or one or more piezoelectric micromachined ultrasonic transducers (PMUTs).

In a non-limiting example, the ultrasonic transducer array 602 may include between approximately 6,000-10,000 (e.g., 8,960) active CMUTs on the chip, forming an array of hundreds of CMUTs by tens of CMUTs (e.g., 140×64). The CMUT element pitch may be between 150-250 um, such as 208 um, and thus, result in the total dimension of between 10-50 mm by 10-50 mm (e.g., 29.12 mm×13.312 mm).

In some embodiments, the TX circuitry 604 may, for example, generate pulses that drive the individual elements of, or one or more groups of elements within, the ultrasonic transducer array(s) 602 so as to generate acoustic signals to be used for imaging. The RX circuitry 606, on the other hand, may receive and process electronic signals generated by the individual elements of the ultrasonic transducer array(s) 602 when acoustic signals impinge upon such elements.

With further reference to FIG. 9, in some embodiments, the timing and control circuit 608 may be, for example, responsible for generating all timing and control signals that are used to synchronize and coordinate the operation of the other elements in the device 600. In the example shown, the timing and control circuit 608 is driven by a single clock signal CLK supplied to an input port 616. The clock signal CLK may be, for example, a high-frequency clock used to drive one or more of the on-chip circuit components. In some embodiments, the clock signal CLK may, for example, be a 1.5625 GHz or 2.5 GHz clock used to drive a high-speed serial output device (not shown in FIG. 9) in the signal conditioning/processing circuit 610, or a 20 Mhz or 40 MHz clock used to drive other digital components on the die 612, and the timing and control circuit 608 may divide or multiply the clock CLK, as necessary, to drive other components on the die 612. In other embodiments, two or more clocks of different frequencies (such as those referenced above) may be separately supplied to the timing and control circuit 608 from an off-chip source.

In some embodiments, the output range of a same (or single) transducer unit in an ultrasound device may be anywhere in a range of 1-12 MHz (including the entire frequency range from 1-12 MHz), making it a universal solution, in which there is no need to change the ultrasound heads or units for different operating ranges or to image at different depths within a patient. That is, the transmit and/or receive frequency of the transducers of the ultrasonic transducer array may be selected to be any frequency or range of frequencies within the range of 1 MHz-12 MHz. The universal device 600 described herein may thus be used for a broad range of medical imaging tasks including, but not limited to, imaging a patient's liver, kidney, heart, bladder, thyroid, carotid artery, lower venous extremity, and performing central line placement. Multiple conventional ultrasound probes would have to be used to perform all these imaging tasks. By contrast, a single universal ultrasound device 600 may be used to perform all these tasks by operating, for each task, at a frequency range appropriate for the task, as shown in the examples of Table 4 together with corresponding depths at which the subject may be imaged.

TABLE 4
Illustrative depths and frequencies at which a ultrasound
device implemented in accordance with embodiments
described herein may image a subject.

| Organ | Frequencies | | Depth (up to) | |
|---|---|---|---|---|
| Liver/Right Kidney | 2-5 | MHz | 15-20 | cm |
| Cardiac (adult) | 1-5 | MHz | 20 | cm |
| Bladder | 2-5 MHz; 3-6 MHz | | 10-15 cm; 5-10 cm | |
| Lower extremity venous | 4-7 | MHz | 4-6 | cm |
| Thyroid | 7-12 | MHz | 4 | cm |
| Carotid | 5-10 | MHz | 4 | cm |
| Central Line Placement | 5-10 | MHz | 4 | cm |

The power management circuit 618 may be, for example, responsible for converting one or more input voltages VIN from an off-chip source into voltages needed to carry out operation of the chip, and for otherwise managing power consumption within the device 600. In some embodiments, for example, a single voltage (e.g., 12V, 80V, 100V, 120V, etc.) may be supplied to the chip and the power management circuit 618 may step that voltage up or down, as necessary, using a charge pump circuit or via some other DC-to-DC voltage conversion mechanism. In other embodiments, multiple different voltages may be supplied separately to the power management circuit 618 for processing and/or distribution to the other on-chip components.

In the embodiment shown above, all of the illustrated elements are formed on a single semiconductor die 612. It should be appreciated, however, that in alternative embodiments one or more of the illustrated elements may be instead located off-chip, in a separate semiconductor die, or in a separate device. Alternatively, one or more of these components may be implemented in a DSP chip, a field programmable gate array (FPGA) in a separate chip, or a separate application specific integrated circuitry (ASIC) chip. Additionally, and/or alternatively, one or more of the components in the beamformer may be implemented in the semiconductor die 612, whereas other components in the beamformer may be implemented in an external processing device in hardware or software, where the external processing device is capable of communicating with the ultrasound device 600.

In addition, although the illustrated example shows both TX circuitry 604 and RX circuitry 606, in alternative embodiments only TX circuitry or only RX circuitry may be employed. For example, such embodiments may be employed in a circumstance where one or more transmission-only devices are used to transmit acoustic signals and one or more reception-only devices are used to receive acoustic signals that have been transmitted through or reflected off of a subject being ultrasonically imaged.

It should be appreciated that communication between one or more of the illustrated components may be performed in any of numerous ways. In some embodiments, for example, one or more high-speed busses (not shown), such as that employed by a unified Northbridge, may be used to allow high-speed intra-chip communication or communication with one or more off-chip components.

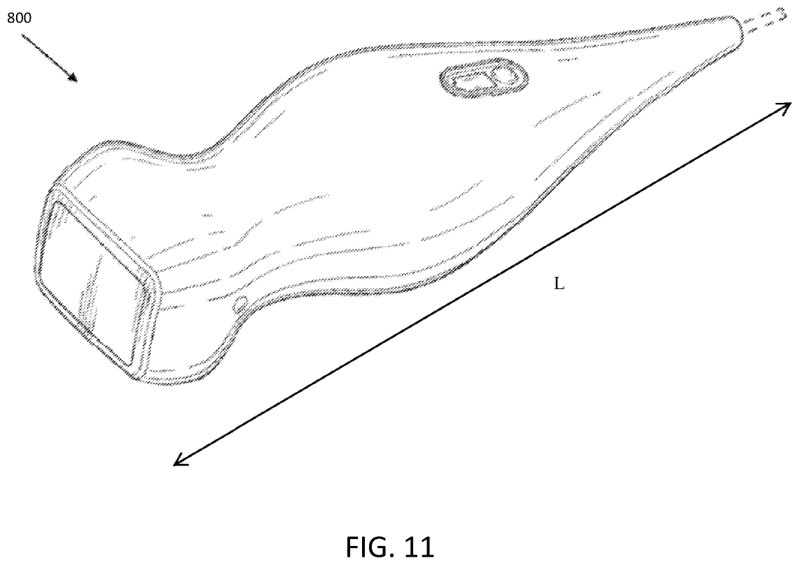

In some embodiments, the ultrasonic transducer elements of the ultrasonic transducer array 602 may be formed on the same chip as the electronics of the TX circuitry 604 and/or RX circuitry 606. The ultrasonic transducer arrays 602, TX circuitry 604, and RX circuitry 606 may be, in some embodiments, integrated in a single ultrasound probe. In some embodiments, the single ultrasound probe may be a hand-held probe including, but not limited to, the hand-held probes described below with reference to FIG. 11.

A CMUT may include, for example, a cavity formed in a CMOS wafer, with a membrane overlying the cavity, and in some embodiments sealing the cavity. Electrodes may be provided to create an ultrasonic transducer cell from the covered cavity structure. The CMOS wafer may include integrated circuitry to which the ultrasonic transducer cell may be connected. The ultrasonic transducer cell and CMOS wafer may be monolithically integrated, thus forming an integrated ultrasonic transducer cell and integrated circuit on a single substrate (the CMOS wafer).

In the example shown, one or more output ports 614 may output a high-speed serial data stream generated by one or more components of the signal conditioning/processing circuit 610. Such data streams may be, for example, generated by one or more USB 3.0 modules, and/or one or more 10 GB, 40 GB, or 100 GB Ethernet modules, integrated on the die 612. It is appreciated that other communication protocols may be used for the output ports 614.

In some embodiments, the signal stream produced on output port 614 can be provided to a computer, tablet, or smartphone for the generation and/or display of two-dimensional, three-dimensional, and/or tomographic images. In some embodiments, the signal provided at the output port 614 may be ultrasound data provided by the one or more beamformer components or auto-correlation approximation circuitry, where the ultrasound data may be used by the computer (external to the ultrasound device) for displaying the ultrasound images. In embodiments in which image formation capabilities are incorporated in the signal conditioning/processing circuit 610, even relatively low-power devices, such as smartphones or tablets which have only a limited amount of processing power and memory available for application execution, can display images using only a serial data stream from the output port 614. As noted above, the use of on-chip analog-to-digital conversion and a high-speed serial data link to offload a digital data stream is one of the features that helps facilitate an “ultrasound on a chip” solution according to some embodiments of the technology described herein.

Devices 600 such as that shown in FIG. 9 may be used in various imaging and/or treatment (e.g., HIFU) applications, and the particular examples described herein should not be viewed as limiting. In one illustrative implementation, for example, an imaging device including an N×M planar or substantially planar array of CMUT elements may itself be used to acquire an ultrasound image of a subject (e.g., a person's abdomen) by energizing some or all of the elements in the ultrasonic transducer array(s) 602 (either together or individually) during one or more transmit phases, and receiving and processing signals generated by some or all of the elements in the ultrasonic transducer array(s) 602 during one or more receive phases, such that during each receive phase the CMUT elements sense acoustic signals reflected by the subject. In other implementations, some of the elements in the ultrasonic transducer array(s) 602 may be used only to transmit acoustic signals and other elements in the same ultrasonic transducer array(s) 602 may be simultaneously used only to receive acoustic signals. Moreover, in some implementations, a single imaging device may include a P×Q array of individual devices, or a P×Q array of individual N×M planar arrays of CMUT elements, which components can be operated in parallel, sequentially, or according to some other timing scheme so as to allow data to be accumulated from a larger number of CMUT elements than can be embodied in a single device 600 or on a single die 612.

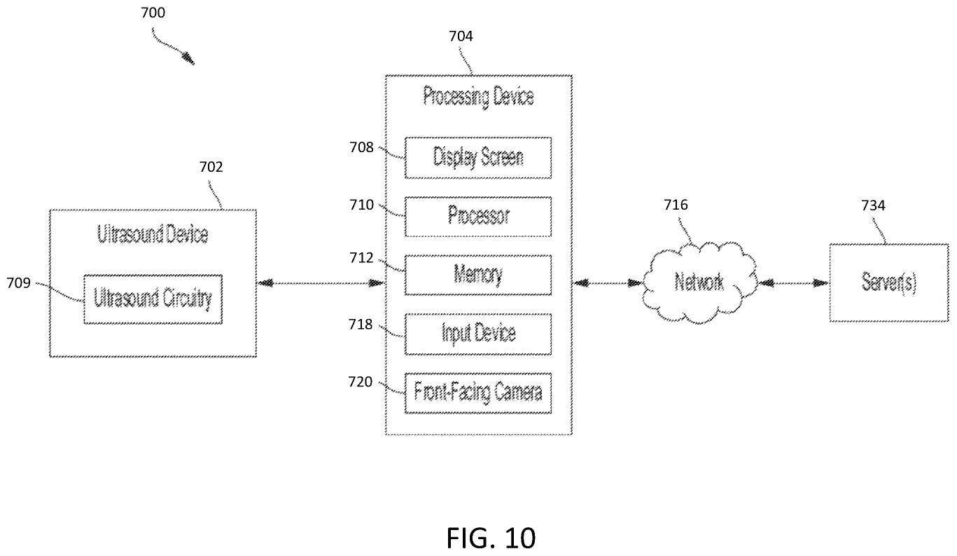

FIG. 10 illustrates a schematic block diagram of an example ultrasound system 700 which may implement various aspects of the technology described herein. In some embodiments, ultrasound system 700 may include an ultrasound device 702, an example of which is implemented in ultrasound device 600. For example, the ultrasound device 702 may be a handheld ultrasound probe. Additionally, the ultrasound system 700 may include a processing device 704, a communication network 716, and one or more servers 734. The ultrasound device 702 may be configured to generate ultrasound data that may be employed to generate an ultrasound image. The ultrasound device 702 may be constructed in any of a variety of ways. In some embodiments, the ultrasound device 702 includes a transmitter that transmits a signal to a transmit beamformer which in turn drives transducer elements within a transducer array to emit pulsed ultrasound signals into a structure, such as a patient. The pulsed ultrasound signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. In some embodiments, the ultrasound device 702 may include an ultrasound circuitry 709 that may be configured to generate the ultrasound data. For example, the ultrasound device 702 may include semiconductor die 612 for implementing the various techniques described in.

Reference is now made to the processing device 704. In some embodiments, the processing device 704 may be communicatively coupled to the ultrasound device 702 (e.g., 600 in FIG. 1) wirelessly or in a wired fashion (e.g., by a detachable cord or cable) to implement at least a portion of the process for approximating the auto-correlation of ultrasound signals. For example, one or more beamformer components (of FIG. 1) may be implemented on the processing device 704. In some embodiments, the processing device 704 may include one or more processing devices (processors) 710, which may include specially-programmed and/or special-purpose hardware such as an ASIC chip. The processor 710 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

In some embodiments, the processing device 704 may be configured to process the ultrasound data received from the ultrasound device 702 to generate ultrasound images for display on the display screen 708. The processing may be performed by, for example, the processor(s) 710. The processor(s) 710 may also be adapted to control the acquisition of ultrasound data with the ultrasound device 702. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

In some embodiments, the processing device 704 may be configured to perform various ultrasound operations using the processor(s) 710 (e.g., one or more computer hardware processors) and one or more articles of manufacture that include non-transitory computer-readable storage media such as the memory 712. The processor(s) 710 may control writing data to and reading data from the memory 712 in any suitable manner. To perform certain of the processes described herein, the processor(s) 710 may execute one or more processor-executable instructions stored in one or more non-transitory computer-readable storage media (e.g., the memory 712), which may serve as non-transitory computer-readable storage media storing processor-executable instructions for execution by the processor(s) 710.

The camera 720 may be configured to detect light (e.g., visible light) to form an image. The camera 720 may be on the same face of the processing device 704 as the display screen 708. The display screen 708 may be configured to display images and/or videos, and may be, for example, a liquid crystal display (LCD), a plasma display, and/or an organic light emitting diode (OLED) display on the processing device 704. The input device 718 may include one or more devices capable of receiving input from a user and transmitting the input to the processor(s) 710. For example, the input device 718 may include a keyboard, a mouse, a microphone, touch-enabled sensors on the display screen 708, and/or a microphone. The display screen 708, the input device 718, the camera 720, and/or other input/output interfaces (e.g., speaker) may be communicatively coupled to the processor(s) 710 and/or under the control of the processor 710.

It should be appreciated that the processing device 704 may be implemented in any of a variety of ways. For example, the processing device 704 may be implemented as a handheld device such as a mobile smartphone or a tablet. Thereby, a user of the ultrasound device 702 may be able to operate the ultrasound device 702 with one hand and hold the processing device 704 with another hand. In other examples, the processing device 704 may be implemented as a portable device that is not a handheld device, such as a laptop. In yet other examples, the processing device 704 may be implemented as a stationary device such as a desktop computer. The processing device 704 may be connected to the network 706 over a wired connection (e.g., via an Ethernet cable) and/or a wireless connection (e.g., over a WiFi network). The processing device 704 may thereby communicate with (e.g., transmit data to or receive data from) the one or more servers 734 over the network 716. For example, a party may provide from the server 734 to the processing device 704 processor-executable instructions for storing in one or more non-transitory computer-readable storage media (e.g., the memory 712) which, when executed, may cause the processing device 704 to perform ultrasound processes. FIG. 10 should be understood to be non-limiting. For example, the ultrasound system 700 may include fewer or more components than shown and the processing device 704 and ultrasound device 702 may include fewer or more components than shown. In some embodiments, the processing device 704 may be part of the ultrasound device 702.

FIG. 11 illustrates an example handheld ultrasound probe, in accordance with certain embodiments described herein. The handheld ultrasound probe 800 may implement the ultrasound device 600 in FIG. 9. The handheld ultrasound probe 800 may be the same or similar to the handheld ultrasound probes described above (e.g., 190, 290, and/or 390). The handheld ultrasound probe 800 may be an ultrasound device or include an ultrasound device, e.g., 600 (FIG. 9) or 702 (FIG. 10) in operative communicative with a processing device (e.g., 704) and transmit the detected signals to the processing device. Alternatively, and/or additionally, the ultrasound probe 800 may include an ultrasound device and a processing device for performing operations over ultrasound signals received from the ultrasonic transducer. In some embodiments, the handheld ultrasound probe 800 may be configured to communicate with the processing device (e.g., 704) wired or wirelessly. Thus, the handheld ultrasound probe 800 may have a suitable dimension and weight. For example, the ultrasound probe 800 may have a cable for wired communication with a processing device, and have a length L about 100 mm-300 mm (e.g., 175 mm) and a weight about 200 grams-500 grams (e.g., 312 g). In another example, the ultrasound probe 800 may be capable of communicating with a processing device wirelessly. As such, the handheld ultrasound probe 800 may have a length about 140 mm and a weight about 265 g. It is appreciated that other dimensions and weight may be possible.

Further description of ultrasound devices and systems may be found in U.S. Pat. No. 9,521,991, the content of which is incorporated by reference herein in its entirety; and U.S. Pat. No. 11,311,274, the content of which is incorporated by reference herein in its entirety.

One or more embodiments of the disclosure may have one or more of the following advantages and improvements over conventional ultrasound probe technologies: improved ergonomic handling and manipulation of handheld ultrasound probes; improving a user's ability to grip and maintain control of a handheld ultrasound probe; reduction of strain and fatigue in a user's hand, wrist, and/or arm while operating a handheld ultrasound probe. Furthermore, each of the above-listed advantages of embodiments of the disclosure may additionally result in: reduction or prevention of injuries, in ultrasound operators or technicians, that are caused by stress or strain from prolonged operation of a handheld ultrasound probe; and enabling patients, especially those patients who are elderly, arthritic, obese, disabled, or otherwise having impaired reach or dexterity to perform ultrasound-based diagnostic tests and imaging on themselves.

Although the disclosure has been described with respect to a limited number of embodiments, those skilled in the art, having benefit of this disclosure, will appreciate that various other embodiments may be devised without departing from the scope of the present disclosure. Accordingly, the scope of the disclosure should be limited only by the attached claims.

What is claimed is:

1. An attachment assembly, for ergonomic handling of a handheld ultrasound probe, the attachment assembly comprising:
a plate-shaped member with a proximal surface and a distal surface, the plate-shaped member comprising a passage between the proximal surface and the distal surface along a first axis, the passage configured to accommodate the handheld ultrasound probe, wherein
the plate-shaped member has a thickness between the proximal surface and the distal surface along the first axis and a width along a second axis perpendicular to the first axis, the thickness being less than the width, and
when the handheld ultrasound probe is inserted in the attachment assembly, a portion of a transducer head of the handheld ultrasound probe protrudes from the distal surface of the plate-shaped member.

2. The attachment assembly according to claim 1, wherein
the attachment assembly is divided into two assembly sections in a plane defined by the first axis and the second axis, and
the assembly sections each comprise one or more fastener holes that extend in a direction perpendicular to the plane,
the assembly sections, when the attachment assembly is attached to the handheld ultrasound probe, are attached to each other by fasteners that are inserted into the fastener holes.

3. The attachment assembly according to claim 2, wherein
the fasteners are one selected from a group consisting of bolts, screws, clips, rivets, springs, and hooks.

4. The attachment assembly according to claim 1, further comprising:
a tapered support member that is rigidly connected to the plate-shaped member and tapers from the proximal surface of the plate-shaped member.

5. The attachment assembly according to claim 1, wherein
the distal surface of the plate-shaped member is substantially flat.

6. The attachment assembly according to claim 1, wherein
the thickness of the plate-shaped member is less than one third of the width of the plate-shaped member.

7. An attachment assembly for ergonomic handling of a handheld ultrasound probe, the attachment assembly comprising:
a shell that attaches to the handheld ultrasound probe and partially covers the handheld ultrasound probe; and
strap anchors that are each connected to the shell and are disposed at opposite ends of the shell.

8. The attachment assembly according to claim 7, further comprising:
a flexible strap that is attached to each of the strap anchors.

9. The attachment assembly according to claim 7, wherein
the strap anchors are protrusions that each extend away from the shell in a direction that is substantially perpendicular to a length direction defined by a distance between the strap anchors, and
each strap anchor comprises a slot.

10. The attachment assembly according to claim 7, wherein
the shell comprises a clamping section with a substantially C-shaped opening, the clamping section partially accommodating the handheld ultrasound probe in the C-shaped opening.

11. The attachment assembly according to claim 10, wherein
the C-shaped opening accommodates greater than 50% of a cross-sectional area of the handheld ultrasound probe.

12. The attachment assembly according to claim 11, wherein
the clamping section is made of a hard-elastic material, and
the C-shaped opening widens when the handheld ultrasound probe is inserted into the attachment assembly.

13. An attachment for ergonomic handling of a handheld ultrasound probe, the attachment comprising:
a curved bar that is continuous between a proximal attachment flange, of the curved bar, that attaches to a first location of the handheld ultrasound probe and a distal attachment flange, of the curved bar, that attaches to a second location of the handheld ultrasound probe, wherein
a segment of the curved bar forms a bar handle.

14. The attachment according to claim 13, wherein
the segment that forms the bar handle is spatially separated from the handheld ultrasound probe to establish an opening between the bar handle and the handheld ultrasound probe.

15. The attachment according to claim 13, wherein
the proximal attachment flange and the distal attachment flange are removably attachable to the handheld ultrasound probe by fasteners.

16. The attachment according to claim 15, wherein
the proximal attachment flange and the distal attachment flange each comprise a fastener plate, that has one or more fastener holes, and
the fasteners are removably inserted into the fastener holes.

17. The attachment according to claim 15, wherein
the fasteners are one selected from a group consisting of bolts, screws, clips, rivets, springs, and hooks.

18. The attachment according to claim 13, wherein
a length of the segment that forms the bar handle extends past the proximal attachment flange, in a proximal direction defined by a distance from the distal attachment flange to the proximal attachment flange.

19. The attachment according to claim 18, further comprising:
a palm handle that is attached to the curved bar and is offset, in the proximal direction, from the proximal attachment flange, wherein
the palm handle extends away from the curved bar in a direction that is perpendicular to the proximal direction.

20. The attachment according to claim 19, wherein
the palm handle has a proximal surface that faces substantially away from the handheld ultrasound probe, and
the proximal surface is curved so as to be substantially convex.
